(19) United States
(12) Patent Application Publication
(10) Pub. No.: US 2005/0089572 A1
(43) Pub. Date: Apr. 28, 2005

(54) Controlled release drug delivery system of pravastatin

(76) Inventors: Manoj Kumar, Gurgaon (IN); Naresh Talwar, New Delhi (IN); Rajeev Singh Raghuvanshi, New Delhi (IN); Ashok Kumar Rampal, Punjab (IN)

Correspondence Address: Jayadeep R Deshmukh;Ranbaxy Inc, Suite 2100, 600 College Road East, Princeton, NJ, 08540, US

(21) Appl. No.: 10/507,513

(22) PCT Filed: Mar. 22, 2002
(86) PCT No.: PCT/IB02/00872

(51) Int. Cl. A61K 31/225; A61K 9/24

(52) U.S. Cl. 424/471; 514/548; 514/460

(57) ABSTRACT

The present invention relates to an oral drug delivery system comprising pravastatin or its pharmaceutically acceptable salts such that the system provides enhanced stability in the acidic environment of the stomach and exhibits controlled release of the drug.

### FIELD OF THE INVENTION

The present invention relates to an oral drug delivery system comprising pravastatin or its pharmaceutically acceptable salts such that the system provides enhanced stability in the acidic environment of the stomach and exhibits controlled release of the drug.

### BACKGROUND OF THE INVENTION

Controlled release dosage forms foster both better patient compliance and decreased incidences of adverse drug reactions. Central to the formulation development of controlled release systems are many variables that influence the in vivo release and subsequent absorption of the active ingredients from the gastrointestinal tract. Therefore, to design an optimum oral controlled release system, it is necessary to take into account the physico-chemical and physiological environment of the gastrointestinal tract.

It is well recognized by those skilled in the art that the systems so designed for sustained or controlled drug delivery functions on the release mechanisms such as dissolution, erosion, diffusion and the like are broadly categorized as osmotic systems, dissolution systems, and diffusion systems. An osmotic system comprises a tablet consisting of a core of drug surrounded by a semi-permeable membrane containing an orifice through which water flows in on exposure to aqueous body fluids due to the generation of osmotic pressure gradient. The drug is released through the orifice at a constant rate which may vary depending upon the drug concentration, orifice diameter, osmotic pressure difference, and the like, until the drug concentration inside the tablet falls below saturation. Dissolution systems are based on the inherent dissolution rate of the drug itself, or of a particular salt or a derivative. Alternatively, the drug is coated with a slow dissolving coating, or incorporated into a slow dissolving carrier.

Diffusion systems include both reservoir devices and matrix devices. In former, core containing the drug is encased by a polymeric membrane wherein the drug release through the membrane is governed by Fick's first law of diffusion. In matrix systems, dissolved or dispersed drug is distributed uniformly throughout an inert polymer matrix and the drug release involves dissolution of the drug from the surface layers, followed by dissolution from the underlying layers.

However, the design of a controlled release formulation for drugs which are susceptible to degradation/transformation in acid media present particular problems for the pharmaceutical formulator. The degradation of such drugs is catalyzed by acidic reacting compounds and it is obvious that an oral dosage form of such drugs must be protected from contact with the acid reacting gastric fluids to hinder degradation in an attempt to improve absorption. The various systems described above lend themselves readily to the formulation of extended release formulations of drugs which are unaffected by pH as they traverse the alimentary canal, but do not provide adequately protected formulations where the drug is acid labile. The rate of release of acid labile drugs from a pharmaceutical dosage form influence the total extent of absorption to the general circulation. The means of achieving a controlled release of acid labile drugs has been a long sought objective as it involves not only the development of an acid stable, bioavailable dosage form but also that provides release controlled from therein. One such acid labile therapeutic agent is Pravastatin.

Pravastatin, chemically known as (+)-(3R, 5R)-3,5-dihydroxy-7-[(1S,2S,6S,8S,8aR)-6-hydroxy-2-methyl-8-[(S)-2-methylbutyryloxy]-1,2,6,7,8,8a-hexahydro-1-naphthyl] heptanoate, and its pharmaceutically acceptable salts has been described in U.S. Pat. No. 4,346,227 which is incorporated herein by reference.

Pravastatin is an HMG-CoA reductase inhibitor which reduces plasma cholesterol levels by inhibiting de novo cholesterol synthesis and increasing the receptor mediated catabolism of low density lipoproteins. The drug exhibits hepatocellular tissue selectivity, with greatest inhibition of cholesterol synthesis occurring in the liver and thereby inhibiting the unwarranted effects on cholesterol synthesis in non-hepatic (peripheral) cells. Its favorable effects on cardiovascular morbidity and total mortality renders it as an effective alternative to currently used HMG-CoA reductase inhibitors for patients with elevated cholesterol levels, multiple risk factors or coronary heart disease.

However, the therapeutic efficacy of any drug depends to a considerable extent on the design of its pharmaceutical formulation. The physico-chemical attributes and bio-pharmacological characteristics account for the formulation of a stable and bioavailable pharmaceutical composition.

Pravastatin sodium is relatively polar and hydrophilic in nature. It is susceptible to heat, light and moisture. It is also sensitive to a low pH environment and is very unstable at pH 3 or less as found in the stomach wherein the hydroxy acids degrade to form lactone and an inactive isomer primarily, 3-α-hydroxy-isopravastatin (Triscari J. et. al; J. Clin. Pharmacol, 35:142 (1995)]. The acid instability of pravastatin reduces its bioavailability and results in degradation of pravastatin following oral administration.

The literature discloses various approaches to obviate problems related to unfavorable absorption characteristics of pravastatin due to its acid sensitivity.

One such approach mentioned in the prior art pertains to the use of agents that are basic in nature and impart alkaline pH. U.S. Pat. No. 5,030,447, for example, describes a stabilized pharmaceutical composition of pravastatin comprising drug, fillers, binders, disintegrants, lubricants and basifying agents to impart a desired pH of at least 9 and preferably about 10 to an aqueous dispersion of said composition. The essence of the invention is to maintain an alkaline environment to combat the low pH sensitivity of the drug. While such an approach may be suitable for enhancing the shelf-life of the drug, however, the local alkaline environment occurring at the site of dissolution of the composition may damage the natural acidic mantle of the alimentary tract especially, in chronic therapies with HMG-CoA reductase inhibitors. Further, the local alkaline environment might get compromised by the acidic pH of the gastric fluids and not be able to provide adequate protection to the acid labile drug.

Other techniques which have been described in the prior art for enhancing the stability of pravastatin include the formulation of “inclusion compounds” by their complexation with agents such as cyclodextrins. WO 99/49896 relates to a composition of sodium pravastatin characterized in that the composition contains β-cyclodextrin as a stabilizer. Cyclodextrin surrounds the drug molecules and prevents its exposure to the acidic environment. As stated and exemplified in the specification, the amount of β-cyclodextrin is advantageously used in the range of 50-5000 weight parts in proportion to 100 weight parts of sodium pravastatin, below which, the drug is insufficiently stabilized and degrades at high humidity and temperature. It is well recognized by those skilled in the art that the desired stability may be achieved by application of such an approach but not without compromising the release of the drug.

Still other techniques are directed towards use of protective coatings to prevent release of acid labile drugs in the stomach. U.S. Pat. No. 5,225,202 discloses an enteric coated pharmaceutical composition of an acid labile medicament in the form of tablet, beadlet, pellet or particle that is enteric coated with neutralized hydroxypropyl methylcellulose phthalate and a plasticizer which affords protection in a low pH environment of 3 or less while release medicament at a pH of 4.5 or higher. It is well known to the formulation scientist that, with time, under ambient conditions, the enteric coating gives an acidic residue which may degrade the drug within the formulation itself and would adversely influence the storage stability of such dosage forms.

As aforementioned, several pharmaceutical compositions have been described which relate to the means to improve the stability, absorption and thus bioavailability profile of pravastatin. However, none of the solutions described above are completely satisfactory.

As aforesaid, one of the requirements for an acceptable pharmaceutical composition is that it must be sufficiently stable so as not to exhibit substantial decomposition of the active ingredient during the time between manufacture of the composition and absorption of the drug in the body. For the purpose, pharmaceutical compositions which include a medicament which is unstable in an acidic environment such as the stomach require an enteric protective coating to arrest the release of the drug in an unfriendly acidic environment. Depending upon the composition and/or thickness, the enteric coatings are resistant to stomach acid for required periods of time before they begin to disintegrate and permit slow release of the drug in the lower stomach or upper part of the small intestines.

In light of the foregoing, the primary object of the present invention is to provide a pharmaceutical composition of an acid labile drug which is stable upon prolonged storage and that provides the desired therapeutic effect while avoiding the heretofore mentioned disadvantages.

### SUMMARY OF THE INVENTION

It is an object of the present invention to provide a pharmaceutical composition which includes an oral controlled drug delivery system of pravastatin or its pharmaceutically acceptable salts that:
(a) includes an enteric coated polymeric core that exhibits controlled release of pravastatin or its pharmaceutically acceptable salts, incorporated therein,
(b) includes a core comprising a polymer that swells upon imbibition of water and regulates the release of pravastatin or its pharmaceutically acceptable salts,
(c) the core is further surrounded by an inert subcoat and an enteric coat that together minimizes acid caused instability to the drug,
(d) delivers the drug at a controlled rate and exhibits reproducibility of release rate into aqueous media at the absorptive regions of gastrointestinal tract, and
(e) provides, as compared to other oral controlled drug delivery systems, increased absorption of a drug which is absorbed largely from the upper parts of the gastrointestinal tract.

It is also an object of the present invention to provide an oral controlled release delivery system that maintains its physical integrity and dimensional stability when in contact with gastrointestinal fluids and achieves the optimal rate of release of pravastatin. It is a further object of the present invention that a therapeutic dose medicament may be incorporated in a therapeutic system without the loss of any of its desirable attributes. The therapeutic system may be prepared either in the form of beads, pellets, granules, tablets or capsules which constitutes an orally administered delivery system capable of controlling release of pravastatin or its pharmaceutically acceptable salts.

In keeping with these objectives the present invention provides a process for the preparation of an oral controlled drug delivery system of pravastatin or its pharmaceutically acceptable salts which effects better stability, readier bioavailability and to such drug delivery system. As embodied and fully described herein, the present invention provides a drug delivery system for oral administration in humans for the controlled release of pravastatin comprising a core comprising therapeutically effective amount of pravastatin or its pharmaceutically acceptable salts and a water swellable polymer, an inert subcoating surrounding the core comprising at least one film forming polymer, and a coating of an enteric polymer over said subcoat, such that the system provides enhanced stability in the acidic environment of the stomach and exhibits controlled release of the drug.

In a particular embodiment, the present invention describes a pharmaceutical composition in the form of pellets, beads or granules for oral administration in humans for the controlled release of pravastatin comprising a core comprising a therapeutically effective amount of pravastatin or its pharmaceutically acceptable salts and a water swellable polymer, an inert subcoating surrounding the core comprising at least one film forming polymer, and a coating of an enteric polymer over said subcoat; incorporated in an oral controlled drug delivery system such that the system provides enhanced stability in the acidic environment of the stomach and exhibits controlled release of the drug.

The present invention also includes a therapeutic system either in the form of beads, pellets, granules, tablets or capsules having an enteric coated polymeric core comprising pravastatin or its pharmaceutically acceptable salts, water swellable polymer and optionally pharmaceutical adjuvants such as swelling agent, diluent and binder. Also, the pharmaceutical composition in solid dosage form may be optionally over coated with a layer comprising pravastatin or its pharmaceutically acceptable salts which exhibits an immediate release of the drug such that the delivery system exhibits a biphasic release profile having an immediate release and controlled release phases.

In a particular embodiment, the present invention describes a pharmaceutical composition in the form of pellets, beads, granules, tablets or capsules for oral administration in humans for the biphasic release of pravastatin comprising a core comprising a therapeutically effective amount of pravastatin or its pharmaceutically acceptable salts and a water swellable polymer, an inert subcoating surrounding the core comprising at least one film forming polymer, a coating of an enteric polymer over said subcoat; over coated with a layer comprising pravastatin or its pharmaceutically acceptable salts which is further coated with a coating of an enteric polymer; such that the system exhibits a biphasic release profile having an immediate release and controlled release phases.

The present invention is directed to a stable delivery system exhibiting controlled release of pravastatin which degrades in a low pH environment but which is protected from doing so by the enteric coating. The enteric coated pharmaceutical composition of the invention provides for the protection of pravastatin at pH less than 3 (such as found in the stomach) but would permit drug release in regions of pH of 4.5 or higher (such as found in the upper intestines).

The present invention relates to a stable delivery system exhibiting controlled release of pravastatin which is attained through a polymeric core that contains water swellable polymer which may be present as a matrix or a coating over the drug core. The polymer swells upon imbibition of water and provides for controlled release of pravastatin. The rate of release of pravastatin from such a system is primarily dependent on rate of water imbibition, resultant rate of swelling of polymer, drug dissolution and diffusion from the matrix or the coat. The core is enteric coated to protect the drug from the unfriendly acidic environment of the stomach. However, most of the enteric coating materials known in the art are acidic in nature and hence may cause chemical instability when in contact with acid labile drugs such as pravastatin. This is especially true under high temperature and humid conditions experienced during coating process. To minimize this acid caused instability, a protective coat or subcoat is applied between the core and the enteric coat. This subcoat physically separates pravastatin from the acidic enteric coat, and hence improves stability of the formulation.

### DETAILED DESCRIPTION OF THE INVENTION

According to the present invention, the core comprises pravastatin or its pharmaceutically acceptable salts as the active ingredient. The amount of the active ingredient is that which is typically administered for a given period of time. This includes a therapeutically effective amount of the drug which is an amount high enough to significantly positively modify the condition to be treated, but low enough to avoid serious side effects (at a reasonable benefit/risk ratio), within the scope of sound medical judgement. Accordingly, pravastatin or its pharmaceutically acceptable salts may be present in an amount from about 5% to about 25% by weight of the total weight of the pharmaceutical composition.

According to the present invention, the core comprises water swellable polymers which regulate the release of pravastatin. The polymers which are amenable to controlled release therapy utilizing the novel therapeutic delivery system of the present invention include any of those suitable for oral administration. The water swellable polymer forming the matrix in accordance with this invention is any such polymer that is non-toxic, swells upon imbibition of water and provides for controlled release of pravastatin. The hydrophilicity of these polymers causes the drug containing matrix to swell upon ingress of water. These water-swellable polymers may be used individually or in combination. Examples of polymers suitable for this invention include the polymers well known in the pharmaceutical art for their release retarding properties and may be selected from the group consisting of polyvinylpyrrolidone, cellulose ethers such as hydroxypropyl methylcelluloses of different grades, hydroxypropyl celluloses of different grades, hydroxyethyl cellulose, methylcellulose, hydroxypropyl ethylcellulose, hydroxyethyl methylcellulose, carboxymethyl cellulose, sodium carboxymethyl cellulose and the like; acrylic polymers such as available as Eudragit RS 30D, Eudragit RL 30D, Eudragit NE 30D, Eudragit RSPO; natural gums such as xanthan gum, karaya gum, locust bean gum, guar gum, gelan gum, gum arabic, tragacanth, carrageenan, pectin, agar, alginic acid, sodium alginate, and the like.

The amount of polymer relative to the drug may vary depending on the release rate desired, nature of the polymers, their physico-chemical characteristics, and other auxiliary components that may be present as the integral part of the composition. Accordingly, the water swellable polymer constitutes at least 0.20% by weight of the total polymeric content of said composition. However, the polymers together may be present in an amount from about 5% to about 40% by weight, and preferably from about 5% to about 25% by weight of the total weight of the pharmaceutical composition.

Optionally, there may also be incorporated into the core of the present invention, other conventional pharmaceutically acceptable auxiliary components known in the art of formulation development such as swelling agent, diluent and binder. It is to be borne in mind, however, that the conventional pharmaceutical auxiliary additives which might adversely affect the desired rate of release of the drug are not suitable for use therein.

The core in accordance with the present invention may contain a swelling agent selected from the class of compounds commonly known as superdisintegrants which absorb large amounts of fluid and causes the hydrated gel matrix to swell significantly thereby assisting in regulating the release profile of pravastatin over a period of time. Examples of swelling agents that may be used in the present invention include cross-linked polyvinylpyrrolidone, cross-linked carboxymethyl cellulose sodium, sodium starch glycolate, and the like. The swelling agent may be present in an amount from about 5% to about 30%, preferably from about 10% to about 20% and more preferably from about 10% to about 15% by weight of the total weight of the composition.

The core may contain one or more of a water soluble and/or water dispersible diluent. Examples of water soluble diluents that may be used in the present invention include, but are not limited to lactose, calcium sulphate, mannitol, dextrates, dextrin, dextrose, sucrose, disodium hydrogen orthophosphate and the like. Water dispersible diluents which refer to water insoluble pharmaceutical excipients that disperse readily in water include, but are not limited to, cellulose based excipients such as microcrystalline cellulose, powdered cellulose, starches such as com starch, pregelatinised starch, clays or clay minerals such as kaolin, bentonite, attapulgite, salts such as calcium carbonate and the like.

According to the present invention the core may also include a binder to provide cohesiveness to the powder mass. The binders commonly known to the pharmaceutical art may be used in the present invention. Examples of the binders are pregelatinised starch, polyvinylpyrrolidone, hydroxypropyl methylcellulose, sodium carboxymethyl cellulose, starch paste, gelatin, xanthan gum, acacia, guar gum, and the like.

The core in accordance to this invention may also contain other conventional pharmaceutical excipients, recognized in the art of pharmaceutical compounding such as pharmaceutical grade magnesium stearate, sodium stearyl fumarate or stearic acid and the like as a glidant, talc and the like as an anti-adherent and silicon dioxide or hydrogenated vegetable oil and the like as a lubricant which form the integral part of the delivery system.

According to the present invention, the cores are coated with an inert subcoat comprising at least one film forming polymer. The subcoat separates the core from the enteric coating polymer(s) containing free carboxyl groups, which otherwise causes degradation/discolouration of pravastatin during the coating process or during storage. The subcoating layer may also serve as a release regulating layer. The film forming polymers for the subcoat is chosen among the pharmaceutically acceptable, inert polymers used for film-coating applications such as, for instance polyethylene glycol, polyvinylpyrrolidone, polyvinyl alcohol, hydroxypropyl cellulose, methylcellulose, ethyl cellulose, hydroxymethyl cellulose, hydroxypropyl methylcellulose, polyvinyl acetal diethylaminoacetate or the like. The subcoating may consist of pharmaceutically acceptable, water soluble or rapidly disintegrating tablet excipients. Ordinary plasticizers colorants, pigments, titanium dioxide, talc and other additives may also be included into the subcoating layer.

According to the present invention, the enteric coating layer is applied on to the subcoated cores. As used herein “enteric coating”, is a polymer material or materials which encases the medicament core. A suitable pH-sensitive enteric polymer is one which dissolves in intestinal juices at the higher pH levels (pH greater than 4.5), such as within the duodenum or small intestine and therefore permit release of pravastatin in the upper portion of the GI tract and not in the stomach. The polymer coating material is selected such that pravastatin is released when the dosage form reaches the small intestine or a region in which the pH is greater than pH 4.5. Preferred coating pH-sensitive materials are those which remain intact in the acidic environment of the stomach, but which disintegrate or dissolve at the pH commonly found in the small intestine of the patient. The pH-solubility behavior of the enteric polymers of the present invention are such that significant dissolution of the enteric polymer coating will not occur until the dosage form has emptied from the stomach while begins to dissolve in an aqueous solution at pH between about 4.5 to about 5.5. As enteric coating polymers, for example, cellulose acetate phthalate, hydroxypropyl methylcellulose phthalate, polyvinyl acetate phthalate, carboxymethyl ethylcellulose, co-polymerized methacrylic acid/methacrylic acid methyl esters such as, for instance, compounds known under the trade name Eudragit L 12.5 or Eudragit L 100 or Eudragit L30D-55 (Rohm Pharma), and the like may be employed.

The enteric coating may also contain a plasticizers such as, although not limited to, diethyl phthalate, triethyl citrate, triacetin, tributyl sebecate, or polyethylene glycol. Optionally, an anti-adherent which is a hydrophobic material such as talc, magnesium stearate or fumed silica may also be incorporated.

According to the present invention, the pharmaceutical composition is prepared either in the form of pellets, granules, beads, tablets or as matrix capsules. The pellet/beads can be prepared using the commonly known techniques as solution/suspension layering over inert core, extrusion and/or spheronisation and also other granulation techniques. Spheronising agents are added to the composition to get uniform spherical granules or pellets. Commonly used spheronisation aids are microcrystalline cellulose (Avicel PH 101 of FMC Corpn. and Emcocel 50M or Emcocel 90M of Mendell), mixture of microcrystalline cellulose and sodium carboxymethyl cellulose (Avicel RC 591 of FMC Corpn.)

According to the present invention, the capsule shell may be of a hard gelatin or a soft gelatin type. Furthermore, capsules made of starch or hydroxypropyl methylcellulose may also be used.

The pharmaceutical composition in accordance to the present invention may be optionally coated with the drug substance, pravastatin or its pharmaceutically acceptable salts, which provides the immediate pulse of the drug release. The coat comprises drug, a film forming polymer and optionally other suitable ingredients for coating including channelling agents, lubricants and plasticizers.

The film forming polymer may be any suitable water soluble polymer that is conventionally used in the art. The polymers which are amenable to the biphasic therapy utilizing the novel therapeutic delivery system of the present invention include any of those suitable for oral administration without compromising on drug release over the stipulated duration of a conventional, immediate release formulation. Examples, include, but not limited to, hydroxypropyl methylcellulose, hydroxypropyl cellulose, hydroxycellulose, carboxymethylcellulose, polyvinylpyrollidone and the like, and mixtures thereof.

The drug coat may optionally include other pharmaceutically acceptable excipients recognized in the art of pharmaceutical coating such as starch, lactose, polyethylene glycol and the like as a channelling agent, talc, colloidal silica, magnesium stearate and the like as lubricants which aid in anti-sticking properties and triethyl citrate, glyceryl monostearate, glyceryl triacetate, acetyltriethylcitrate, dibutyl phthalate, dibutyl sebacate, ethylene glycol and the like as plasticizers that increase flexibility and toughness of the coat by internally modifying or solvating polymer molecules.

The pellets, granules, beads, tablets or matrix capsules may be coated by fluid-bed coating, pan coating or other standard coating procedures using standard techniques and equipment known to those skilled in the art. The precise conditions for forming and coating composition will vary with the particular apparatus selected and are apparent to the artisan without the need for undue experimentation.

The present invention is illustrated below by reference to the following examples which set forth particularly preferred embodiments. However, it should be noted that these embodiments are illustrative and are not to be construed as limiting the invention in any way.

### EXAMPLE 1

This example illustrates the process for the preparation of controlled release tablets of pravastatin that delivers dual release of the drug showing immediate and controlled release phases. The pharmaceutical composition is given below.

CORE

| | |
|---|---|
| Pravastatin Sodium | 24 g |
| Calcium Carbonate | 50 g |
| Hydroxypropyl methyl cellulose | 60 g |
| (K 100 LVCR) | |
| Sodium Stearyl Fumarate | 6 g |
| Lactose | 160 g |

SUBCOAT

| | |
|---|---|
| Hydroxypropyl methyl cellulose (E-5) | 126 g |
| Talc | 19 g |
| Isopropyl Alcohol | 1000 g |
| Water | 200 g |

ENTERIC COAT

| | |
|---|---|
| Hydroxypropyl methyl cellulose | 110 g |
| pthalate (HP50) | |
| Triethyl citrate | 25 g |
| Talc | 28 g |
| Water | 650 g |
| Ammonia Solution | q.s. |

DRUG LAYERING

| | |
|---|---|
| Pravastatin sodium | 40 g |
| Crosslinked polyvinylpyrrolidone | 10 g |
| (Kollidon CLM) | |
| Polyvinylpyrrolidone (K30) | 2 g |
| Disodium Hydrogen orthophosphate | 0.75 g |
| Water | 110 g |

The tablets were tested for drug release in pH 6.8 phosphate buffer media using USP apparatus 1 with basket speed at 50 rpm. The samples of the media were periodically withdrawn and spectrophotometrically analyzed for pravastatin sodium content. The dissolution results are given in Table 1.

TABLE 1

| | PERCENT PRAVASTATIN |
|---|---|
| TIME (HRS) | RELEASED |
| 1 | 49 |
| 2 | 61 |
| 3 | 73 |
| 4 | 83 |
| 5 | 100 |

### EXAMPLE 2

This example illustrates the process for the preparation of controlled release beads of pravastatin the pharmaceutical composition of which is given below.

CORE
Drug layer over inert seeds having the following composition

| | |
|---|---|
| Pravastatin sodium | 40 g |
| Crosslinked polyvinylpyrrolidone | 10 g |
| (Kollidon CLM) | |
| Polyvinylpyrrolidone (K30) | 2 g |
| Disodium Hydrogen orthophosphate | 0.75 g |
| Water | 150 g |

SUBCOAT

Ethyl cellulose (Surelease)

ENTERIC COAT

| | |
|---|---|
| Hydroxypropyl methyl cellulose | 20 g |
| Pthalate (HP50) | |
| Triethyl citrate | 4.5 g |
| Talc | 5.1 g |
| Water | 120 g |
| Ammonia Solution | q.s. |

The beads were characterized for drug release in pH 6.8 phosphate buffer as described in Example 1 and the dissolution results are recorded in Table 2.

TABLE 2

| | PERCENT PRAVASTATIN |
|---|---|
| TIME (HRS) | RELEASED |
| 1 | 29 |
| 2 | 77 |
| 3 | 86 |
| 4 | 96 |
| 6 | 100 |

### EXAMPLE 3

This example illustrates the process for the preparation of controlled release tablets of pravastatin that delivers dual release of the drug showing immediate and controlled release phases. The over coat of the drug exhibiting immediate release characteristics was coated with an enteric polymer to provide adequate protection in the low gastric pH. The pharmaceutical composition is given below.

CORE

| | |
|---|---|
| Pravastatin Sodium | 24 g |
| Calcium Carbonate | 50 g |
| Hydroxypropyl methyl cellulose | 60 g |
| (K 100 LVCR) | |
| Sodium Stearyl Fumarate | 6 g |
| Lactose | 160 g |

SUBCOAT

| | |
|---|---|
| Hydroxypropyl methyl cellulose (E-5) | 126 g |
| Talc | 18.9 g |
| Isopropyl Alcohol | 1020 g |
| Water | 180 g |

ENTERIC COAT

| | |
|---|---|
| Hydroxypropyl methyl cellulose | 110 g |
| pthalate (HP50) | |
| Triethyl citrate | 24.44 g |
| Talc | 28.12 g |
| Water | 660 g |
| Ammonia Solution | q.s. |

DRUG LAYERING

| | |
|---|---|
| Pravastatin sodium | 40 g |
| Crosslinked polyvinylpyrrolidone | 10 g |
| (Kollidon CLM) | |
| Polyvinylpyrrolidone (K30) | 2 g |
| Disodium Hydrogen orthophosphate | 0.75 g |
| Water | 110 g |

SUBCOAT

| | |
|---|---|
| Hydroxypropyl methyl cellulose (E-5) | 126 g |
| Talc | 18.9 g |
| Isopropyl Alcohol | 1020 g |
| Water | 180 g |

ENTERIC COAT

| | |
|---|---|
| Hydroxypropyl methyl cellulose | 110 g |
| pthalate (HP50) | |
| Triethyl citrate | 24.44 g |
| Talc | 28.12 g |
| Water | 660 g |
| Ammonia Solution | q.s. |

The tablets were characterized for drug release in pH 6.8 phosphate buffer as described in Example 1 and the dissolution results are given in Table 3.

TABLE 3

| | PERCENT PRAVASTATIN |
|---|---|
| TIME (HRS) | RELEASED |
| 1 | 41 |
| 2 | 50 |
| 3 | 66 |
| 4 | 81 |
| 5 | 97 |
| 6 | 102 |

While this invention has been described with an emphasis upon preferred embodiments, It will be obvious to those of ordinary skill in the art that variations in the preferred methods of the present invention may be used and that it is intended that the invention may be practiced otherwise than as specifically described herein. Accordingly, this invention includes all modifications encompassed within the spirit and scope of the invention as defined by the following claims

1. A drug delivery system for oral administration in humans for the controlled release of pravastatin comprising:
(a) a core comprising a therapeutically effective amount of pravastatin or its pharmaceutically acceptable salts and a water swellable polymer;
(b) an inert subcoating surrounding the core comprising at least one film forming polymer; and
(c) a coating of an enteric polymer over said subcoating;
such that the system provides enhanced stability in the acidic environment of the stomach and exhibits controlled release of the drug.

2. The drug delivery system according to claim 1 wherein pravastatin or its pharmaceutically acceptable salts comprises from about 5% to about 25% by weight of the total weight of the composition.

3. The drug delivery system according to claim 1 wherein said water swellable polymer is selected from the group consisting of pyrrolidone, cellulose ether, acrylic polymer, natural gum, and mixtures thereof.

4. The drug delivery system according to claim 3 wherein the pyrrolidone is polyvinylpyrrolidone.

5. The drug delivery system according to claim 3 wherein the cellulose ether is selected from the group consisting of hydroxypropyl cellulose, hydroxypropyl methylcellulose, hydroxyethyl cellulose, methylcellulose, hydroxyethyl methylcellulose, hydroxypropyl, ethylcellulose, carboxymethyl cellulose, sodium carboxymethyl cellulose, hydroxycellulose, and mixtures thereof.

6. The drug delivery system according to claim 3 wherein the acrylic polymer is selected from the group consisting of methacrylates, polyacrylates copolymers, and mixtures thereof.

7. The drug delivery system according to claim 3 wherein the natural gum is selected from the group consisting of xanthan gum, karaya gum, locust bean gum, guar gum, gelan gum, gum arabic, tragacanth, carrageenan, pectin, agar, alginic acid, sodium alginate, and mixtures thereof.

8. The drug delivery system according to claim 1 wherein the water swellable polymer comprises from about 5% to about 40% by weight of the total weight of the composition.

9. The drug delivery system according to claim 8 wherein the water swellable polymer comprises from about 5% to about 25% by weight of the total weight of the composition.

10. The drug delivery system according to claim 1 wherein the core further comprises swelling agents.

11. The drug delivery system according to claim 10 wherein the swelling agent comprises a superdisintegrant.

12. The drug delivery system according to claim 11 wherein the swelling agent is selected from the group consisting of cross-linked polyvinylpyrrolidone, cross-linked sodium carboxymethyl cellulose, sodium starch glycolate, and mixtures thereof.

13. The drug delivery system according to claim 10 wherein the swelling agent comprises from about 5% to about 30% by weight of the total weight of the composition.

14. The drug delivery system according to claim 10 wherein the core further comprises diluents, binder, glidant, anti-adherent, lubricant, or mixtures thereof.

15. The drug delivery system according to claim 1 wherein the subcoat comprising a film forming polymer is selected from the group consisting of polyethylene glycol, polyvinylpyrrolidone, polyvinyl alcohol, hydroxypropyl cellulose, methylcellulose, ethyl cellulose, hydroxymethyl cellulose, hydroxypropyl methylcellulose, polyvinyl acetal diethylaminoacetate, and mixtures thereof.

16. The drug delivery system according to claim 1 wherein the enteric polymer is selected from the group consisting of cellulose acetate phthalate, hydroxypropyl methylcellulose phthalate, polyvinyl acetate phthalate, carboxymethyl ethylcellulose, co-polymerized methacrylic acid/methacrylic acid methyl esters and mixtures thereof.

17. The drug delivery system according to claim 1 wherein the subcoating and/or enteric coating may further comprise plasticizer, anti-adherent, colorant, and mixtures thereof.

18. A drug delivery system for oral administration in humans for the biphasic release of pravastatin comprising:
(a) a core comprising therapeutically effective amount of pravastatin or its pharmaceutically acceptable salts and a water swellable polymer;
(b) an inert subcoating surrounding the core comprising at least one film forming polymer;
(c) a coating of an enteric polymer over said subcoat;
(d) an overcoat of pravastatin or its pharmaceutically acceptable salts over the enteric coat; and
(e) a coating of an enteric polymer over said drug overcoat;
such that the system exhibits a biphasic release profile having an immediate release and controlled release phases.

19. The drug delivery system according to claims 1 or 18 wherein the dosage form being formed into a physical form selected from the group consisting of pellets, beads, granules, tablets and capsules.

20. The drug delivery system according to claim 19 wherein the capsule shell is made of gelatin, hydroxypropyl methylcellulose or starch.

21. The drug delivery system according to claim 19 wherein tablet dosage forms further comprises coating with a fast dissolving film of a water soluble polymer.

22. A drug delivery system for oral administration in humans for the controlled release of pravastatin comprising pravastatin or its pharmaceutically acceptable salts wherein the drug delivery system exhibits the following in vitro dissolution profile when measured in a type 1 dissolution apparatus according to U.S. Pharmacopoeia XXII in pH 6.8 phosphate buffer media at 50 rpm:
(a) more than 20% of the total pravastatin is released within 1 hour of measurement in said apparatus;
(b) more than 50% of the total pravastatin is released within 3 hours of measurement in said apparatus; and
(c) more than 70% of the total pravastatin is released within about 4-6 hours of measurement in said apparatus.
